(19) United States
(12) Patent Application Publication
(10) Pub. No.: US 2004/0220204 A1
(43) Pub. Date: Nov. 4, 2004

(54) Pharmaceutical composition comprising a tricyclic compound for the prevention or treatment of skin diseases

(76) Inventors: Satoshi Ueda, Osaka (JP); Toshiro Sakai, Osaka (JP); Erika Yoshida, Osaka (JP)

Correspondence Address: LEYDIG VOIT & MAYER, LTD, 700 THIRTEENTH ST. NW, SUITE 300, WASHINGTON, DC, 20005-3960, US

(21) Appl. No.: 10/478,635

(22) PCT Filed: May 23, 2002
(86) PCT No.: PCT/JP02/05030

(30) Foreign Application Priority Data
May 28, 2001 (AU) PR 5297

(51) Int. Cl. A61K 31/4745

(52) U.S. Cl. 514/291

(57) ABSTRACT

To provide a pharmaceutical composition comprising a tricyclic compound (I) or its pharmaceutically acceptable salt; monohydric alcohol fatty acid esters; dibasic acid diesters; lower alkylene carbonates; butylene glycol; diethylene glycol mono(lower)alkyl ethers; and thickeners. It is satisfactory in stability and absorption kinetics and/or a low irritation potential.

### TECHNICAL FIELD

This invention relates to a pharmaceutical composition containing tricyclic compound, said composition being stable and having very satisfactory absorption kinetics and/or a low irritation potential. This composition finds application in the therapy and prophylaxis of various diseases of the skin.

### BACKGROUND ART

The tricyclic compound and its pharmaceutically acceptable salt for use in accordance with this invention, is known to have excellent immunosuppressive activity, antimicrobial activity and other pharmacological activities and, as such, be of value for the treatment or prevention of rejection reactions by transplantation of organs or tissues, graft-vs.-host diseases, autoimmune diseases, and infectious diseases [EP-A-0184162, EP-A-0323042, etc.].

Particularly, FK506 Substance among such tricyclic compound (I), which has been shown to be useful for the therapy and prevention of graft rejection in organ transplantation due to its quite excellent immunosuppressive activity.

It is mentioned in EP-A-0315978 that an ethanol solution of FK506 Substance is effective in arresting inflammatory reactions and that FK506 Substance can be provided in the form of a lotion, a gel or a cream. However, there is no specific disclosure of such dosage forms.

Meanwhile, EP-A-0474126 discloses an ointment comprising FK506 Substance and its analogs, a dissolution/absorption promoter added in a sufficient amount to dissolve the active compound, and an ointment base.

Further, WO94/28894 discloses a lotion comprising FK506 Substance and its analogs, a dissolution/absorption promoter, a liquid base, and, optionally, an emulsifier and/or a thickener.

And further, WO99/55332 discloses a pharmaceutical composition comprising macrolide compound, a dissolution/absorption promoter and a pharmaceutical base, and optionally a compatibilizing agent and/or a thickener.

### DISCLOSURE OF INVENTION

The inventors of this invention explored in earnest for a pharmaceutical composition suited for the administration of a tricyclic compound, a representative of which is FK506 Substance, and discovered a dosage form having very satisfactory characteristics, namely stability, good percutaneous absorption and/or low skin irritation potential. Thus, the present invention specifically relates to a gel preparation comprising the tricyclic compound for external application.

In accordance with this invention there is provided a pharmaceutical composition comprising the tricyclic compound (I) or its pharmaceutically acceptable salt;

monohydric alcohol fatty acid esters;

dibasic acid diesters;

lower alkylene carbonates;

butylene glycol;

diethylene glycol mono(lower)alkyl ethers; and

thickeners.

An example of the tricyclic compound of the following formula (I) can be exemplified.

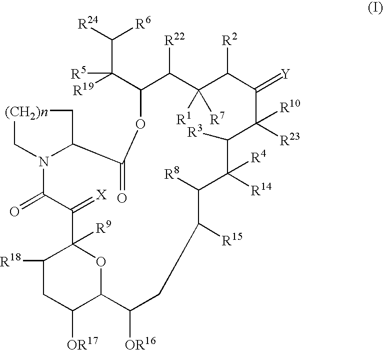

(wherein each of adjacent pairs of R¹ and R², R³ and R⁴ and R⁵ and R⁶ independently

(a) is two adjacent hydrogen atoms, but R² may also be an alkyl group or

(b) may form another bond formed between the carbon atoms to which they are attached;

R⁷ is a hydrogen atom, a hydroxy group, a protected hydroxy group, or an alkoxy group, or an oxo group together with R¹;

R⁸ and R⁹ are independently a hydrogen atom or a hydroxy group;

R¹⁰ is a hydrogen atom, an alkyl group, an alkyl group substituted by one or more hydroxy groups, an alkenyl group, an alkenyl group substituted by one or more hydroxy groups, or an alkyl group substituted by an oxo group;

X is an oxo group, (a hydrogen atom and a hydroxy group), (a hydrogen atom and a hydrogen atom), or a group represented by the formula —CH₂O—;

Y is an oxo group, (a hydrogen atom and a hydroxy group), (a hydrogen atom and a hydrogen atom), or a group represented by the formula N—NR¹¹R¹² or N—OR¹³;

R¹¹ and R¹² are independently a hydrogen atom, an alkyl group, an aryl group or a tosyl group;

R¹³, R¹⁴, R¹⁵, R¹⁶, R¹⁷, R¹⁸, R¹⁹, R²² and R²³ are independently a hydrogen atom or an alkyl group;

R²⁴ is an optionally substituted ring system which may contain one or more heteroatoms;

n is an integer of 1 or 2; and

in addition to the above definitions, Y, R¹⁰ and R²³, together with the carbon atoms to which they are attached, may represent a saturated or unsaturated 5- or 6-membered nitrogen, sulfur and/or oxygen containing heterocyclic ring optionally substituted by one or more groups selected from the group consisting of an alkyl, a hydroxy, an alkoxy, a benzyl, a group of the formula —CH₂Se(C₆H₅), and an alkyl substituted by one or more hydroxy groups.

The definitions used in the above general formula (I) and the specific and preferred examples thereof are now explained and set forth in detail.

The term “lower” means, unless otherwise indicated, a group having 1 to 6 carbon atoms.

Preferable examples of the “alkyl groups” and an alkyl moiety of the “alkoxy group” include a straight or branched chain aliphatic hydrocarbon residue, for example, a lower alkyl group such as methyl, ethyl, propyl, isopropyl, butyl, isobutyl, pentyl, neopentyl and hexyl.

Preferable examples of the “alkenyl groups” include a straight or branched chain aliphatic hydrocarbon residue having one double-bond, for example, a lower alkenyl group such as vinyl, propenyl (e.g., allyl group), butenyl, methylpropenyl, pentenyl and hexenyl.

Preferable examples of the “aryl groups” include phenyl, tolyl, xylyl, cumenyl, mesityl and naphthyl.

Preferable protective groups in the “protected hydroxy groups” and the “protected amino” are 1-(lower alkylthio)-(lower)alkyl group such as a lower alkylthiomethyl group (e.g., methylthiomethyl, ethylthiomethyl, propylthiomethyl, isopropylthiomethyl, butylthiomethyl, isobutylthiomethyl, hexylthiomethyl, etc.), more preferably C₁-C₄ alkylthiomethyl group, most preferably methylthiomethyl group;

trisubstituted silyl group such as a tri(lower)alkylsilyl (e.g., trimethylsilyl, triethylsilyl, tributylsilyl, tert-butyldimethylsilyl, tri-tert-butylsilyl, etc.) or lower alkyl-diarylsilyl (e.g., methyldiphenylsilyl, ethyldiphenylsilyl, propyldiphenylsilyl, tert-butyldiphenylsilyl, etc.), more preferably tri (C₁-C₄)alkylsilyl group and C₁-C₄ alkyldiphenylsilyl group, most preferably tert-butyldimethylsilyl group and tert-butyldiphenylsilyl group; and an acyl group such as an aliphatic, aromatic acyl group or an aliphatic acyl group substituted by an aromatic group, which are derived from a carboxylic acid, sulfonic acid or carbamic acid.

Examples of the aliphatic acyl groups include a lower alkanoyl group optionally having one or more suitable substituents such as carboxy, e.g., formyl, acetyl, propionyl, butyryl, isobutyryl, valeryl, isovaleryl, pivaloyl, hexanoyl, carboxyacetyl, carboxypropionyl, carboxybutyryl, carboxyhexanoyl, etc.; a cyclo(lower)alkoxy(lower)alkanoyl group optionally having one or more suitable substituents such as lower alkyl, e.g., cyclopropyloxyacetyl, cyclobutyloxypropionyl, cycloheptyloxybutyryl, menthyloxyacetyl, menthyloxypropionyl, menthyloxybutyryl, menthyloxypentanoyl, menthyloxyhexanoyl, etc.; a camphorsulfonyl group; or a lower alkylcarbamoyl group having one or more suitable substituents such as carboxy or protected carboxy, for example, carboxy(lower)alkylcarbamoyl group (e.g., carboxymethylcarbamoyl, carboxyethylcarbamoyl, carboxypropylcarbamoyl, carboxybutylcarbamoyl, carboxypentylcarbamoyl, carboxyhexylcarbamoyl, etc.), tri-(lower)alkylsilyl(lower)alkoxycarbonyl(lower)alkylc arbamoyl group (e.g., trimethylsilylmethoxycarbonylethylcarbamoyl, trimethylsilylethoxycarbonylpropylcarbamoyl, triethylsilylethoxycarbonylpropylcarbamoyl, tert-butyldimethylsilylethoxycarbonylpropylcarbamoyl, tri-methylsilylpropoxycarbonylbutylcarbamoyl, etc.) and so on.

Examples of the aromatic acyl groups include an aroyl group optionally having one or more suitable substituents such as nitro, e.g., benzoyl, toluoyl, xyloyl, naphthoyl, nitrobenzoyl, dinitrobenzoyl, nitronaphthoyl, etc.; and an arenesulfonyl group optionally having one or more suitable substituents such as halogen, e.g., benzenesulfonyl, toluenesulfonyl, xylenesulfonyl, naphthalenesulfonyl, fluorobenzenesulfonyl, chlorobenzenesulfonyl, bromobenzenesulfonyl, iodobenzenesulfonyl, etc.

Examples of the aliphatic acyl groups substituted by an aromatic group include ar (lower) alkanoyl group optionally having one or more suitable substituents such as lower alkoxy or trihalo(lower)alkyl, e.g., phenylacetyl, phenylpropionyl, phenylbutyryl, 2-trifluoromethyl-2-methoxy-2-phenylacetyl, 2-ethyl-2-trifluoromethyl-2-phenylacetyl, 2-trifluoromethyl-2-propoxy-2-phenylacetyl, etc.

More preferable acyl groups among the aforesaid acyl groups are C₁-C₄ alkanoyl group optionally having carboxy, cyclo(C₅-C₆)alkoxy(C₁-C₄)alkanoyl group having two (C₁-C₄) alkyls at the cycloalkyl moiety, camphorsulfonyl group, carboxy-(C₁-C₄) alkylcarbamoyl group, tri(C₁-C₄)alkylsilyl(C₁-C₄)alkoxycarbonyl(C₁-C₄)-alkylcarbamoyl group, benzoyl group optionally having one or two nitro groups, benzenesulfonyl group having halogen, or phenyl(C₁-C₄)alkanoyl group having C₁-C₄ alkoxy and trihalo(C₁-C₄)alkyl group. Among these, the most preferable ones are acetyl, carboxypropionyl, menthyloxyacetyl, camphorsulfonyl, benzoyl, nitrobenzoyl, dinitrobenzoyl, iodobenzenesulfonyl and 2-trifluoromethyl-2-methoxy-2-phenylacetyl.

Preferable examples of the “5- or 6-membered nitrogen, sulfur and/or oxygen containing heterocyclic ring” include a pyrrolyl group and a tetrahydrofuryl group.

R²⁴ is an optionally substituted ring system which may contain one or more heteroatoms, Preferable R²⁴ may be cyclo(C₅₋₇)alkyl group optionally having suitable substituents, and the following ones can be exemplified.

(a) a 3,4-di-oxo-cyclohexyl group;

(b) a 3-R²⁰-4-R²¹-cyclohexyl group,

in which R²⁰ is hydroxy, an alkoxy group, an oxo group, or a —OCH₂OCH₂CH₂OCH₃ group, and

R²¹ is hydroxy, —OCN, an alkoxy group, a heteroaryloxy which may be substituted by suitable substituents, 1- or 2-tetrazolyl, a —OCH₂OCH₂CH₂OCH₃ group, a protected hydroxy group, chloro, bromo, iodo, aminooxalyloxy, an azido group, p-tolyloxythiocarbonyloxy, or R²⁵R²⁶CHCOO—,

in which R²⁵ is optionally protected hydroxy or protected amino, and

R²⁶ is hydrogen or methyl, or

R²⁰ and R²¹ together form an oxygen atom in an epoxide ring; or

(c) cyclopentyl group substituted by methoxymethyl, optionally protected hydroxymethyl, acyloxymethyl

(in which the acyl moiety optionally contains either a dimethylamino group which may be quaternized, or a carboxy group which may be esterified), one or more amino and/or hydroxy groups which may be protected, or aminooxalyloxymethyl. A preferred example is a 2-formyl-cyclopentyl group.

“A heteroaryl which may be substituted by suitable substituents” moiety of the “heteroaryloxy which may be substituted by suitable substituents” may be the ones exemplified for R¹ of the compound of the formula of EP-A-532,088, with preference given to 1-hydroxyethylindol-5-yl, the disclosure of which is incorporated herein by reference.

The ticyclic compounds (I) and its pharmaceutically acceptable salt for use in accordance with this invention are well known to have excellent immunosuppressive activity, antimicrobial activity and other pharmacological activities and, as such, be of value for the treatment or prevention of rejection reactions by transplantation of organs or tissues, graft-vs-host diseases, autoimmune diseases, and infectious diseases [EP-A-0184162, EP-A-0323042, EP-A-423714, EP-A-427680, EP-A-465426, EP-A-480623, EP-A-532088, EP-A-532089, EP-A-569337, EP-A-626385, WO89/05303, WO93/05058, WO96/31514, WO91/13889, WO91/19495, WO93/04680, WO93/5059, etc.], the disclosures of which are incorporated herein by reference.

Particularly, the compounds which are designated as FR900506 (=FK506), FR900520 (ascomycin), FR900523, and FR900525 are products produced by microorganisms of the genus Streptomyces, such as Streptomyces tsukubaensis No. 9993 [deposited with National Institute of Bioscience and Human Technology Agency of Industrial Science and Technology (formerly Fermentation Research Institute Agency of Industrial Science and Technology), at 1-3, Higashi 1-chome, Tsukuba-shi, Ibaraki, Japan, date of deposit Oct. 5, 1984, accession number FERM BP-927] or Streptomyces hygroscopicus subsp. yakushimaensis No. 7238 [deposited with National Institute of Bioscience and Human Technology Agency of Industrial Science and Technology (formerly Fermentation Research Institute Agency of Industrial Science and Technology), at 1-3, Higashi 1-chome, Tsukuba-shi, Ibaraki, Japan, date of deposit Jan. 12, 1985, accession number FERM BP-928] [EP-A-0184162]. The FK506 (general name: tacrolimus) of the following chemical formula, in particular, is a representative compound.

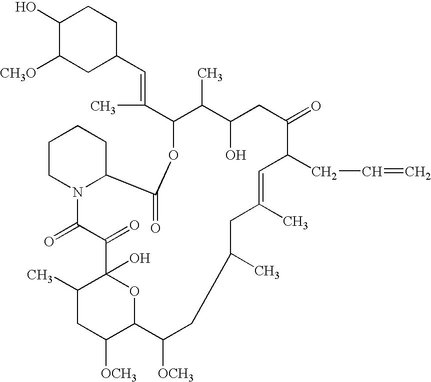

Chemical name:

17-allyl-1,14-dihydroxy-12-[2-(4-hydroxy-3-methoxycyclohexyl)-1-methylvinyl]-23,25-dimethoxy-13,19,21,27-tetramethyl-11,28-dioxa-4-azatricyclo[22.3.1.0^{4,9}]octacos-18-ene-2,3,10,16-tetra one

The preferred examples of the tricyclic compounds (I) are the ones, wherein each of adjacent pairs of R³ and R⁴ or R⁵ and R⁶ independently form another bond formed between the carbon atoms to which they are attached;

each of R⁸ and R²³ is independently a hydrogen atom;

R⁹ is a hydroxy group;

R¹⁰ is a methyl group, an ethyl group, a propyl group or an allyl group;

X is (a hydrogen atom and a hydrogen atom) or an oxo group;

Y is an oxo group;

each of R¹⁴ , R¹⁵, R¹⁶, R¹⁷, R¹⁸, R¹⁹, and R²² is a methyl group;

R²⁴ is a 3-R²⁰-4-R²¹-cyclohexyl group,

in which R²⁰ is hydroxy, an alkoxy group, an oxo group, or a —OCH₂OCH₂CH₂OCH₃ group, and

R²¹ is hydroxy, —OCN, an alkoxy group, a heteroaryloxy which may be substituted by suitable substituents, 1- or 2-tetrazolyl, a —OCH₂OCH₂CH₂OCH₃ group, a protected hydroxy group, chloro, bromo, iodo, aminooxalyloxy, an azido group, p-tolyloxythiocarbonyloxy, or R²⁵R²⁶ CHCOO—,

in which R²⁵ is optionally protected hydroxy or protected amino, and

R²⁶ is hydrogen or methyl, or

R²⁰and R²¹ together form an oxygen atom in an epoxide ring; and

n is an integer of 1 or 2.

The most preferable tricyclic compounds (I) is, in addition to FK506, ascomycin derivatives such as halogenated-ascomycin (e.g., 33-epi-chloro-33-desoxyascomycin), which is disclosed in EP 427,680, example 66a.

The tricyclic compounds(I) maybe in a form of its salt, which includes conventional non-toxic and pharmaceutically acceptable salt such as the salt with inorganic or organic bases, specifically, an alkali metal salt such as sodium salt and potassium salt, an alkali earth metal salt such as calcium salt and magnesium salt, an ammonium salt and an amine salt such as triethylamine salt and N-benzyl-N-methylamine salt.

With respect to the tricyclic compound (I) used in the present invention, it is to be understood that there may be conformers and one or more stereoisomers such as optical and geometrical isomers due to asymmetric carbon atom(s) or double bond(s), and such conformers and isomers are also included within the scope of tricyclic compound (I) in the present invention. And further, the tricyclic compound(I) can be in the form of a solvate, which is included within the scope of the present invention. The solvate preferably include a hydrate and an ethanolate.

Monohydric alcohol fatty acid esters, dibasic acid diesters and lower alkylene carbonates for use in this invention are not particularly restricted provided that they are capable of dissolving tricyclic compound (I) or its pharmaceutically acceptable salt therein and/or promoting its percutaneous absorption. For example, the following examples can be used with advantage.

Monohydric alcohol fatty acid esters:

isopropylmyristate, ethylmyristate, butylmyristate, isocetylmyristate, octyldodecylmyristate, isopropyl palmitate, isostearyl palmitate, isopropyl isostearate, isocetyl isostearate, butyl stearate, isocetyl stearate, cetyl isooctanotate, ethyl linoleate, isopropyl linoleate, hexyl laurate, ethyl oleate, decyl oleate, oleyl oleate, octyldodecyl myristate, hexyldecyl dimethyloctanoate, octyldodecyl neodecanotate, etc.

Among them, isopropyl myristate is the most preferable.

Dibasic acid diesters

diisopropyl adipate, dimethyl adipate, diethyl adipate, diethyl sebacate, diisopropyl sebacate, dipropyl sebacate, diethyl phthalate, diethyl pimelate, etc.

Among them, diisopropyl adipate, diethyl sebacate or their combination are the most preferable ones.

Lower alkylene carbonates

propylene carbonate, ethylene carbonate, etc. The most preferable one is propylene carbonate.

Each amount of said monohydric alcohol fatty acid esters, dibasic acid diesters and lower alkylene carbonates in the composition is not particularly restricted but should be large enough to, dissolve the tricyclic compound (I) and/or promote its percutaneous absorption. For example, each amount thereof is preferably 1˜30% (w/w), more preferably 2˜20% (w/w), still more preferably 3˜15% (w/w).

And more particularly, the total amount of said monohydric alcohol fatty acid esters, dibasic acid diesters and lower alkylene carbonates in the composition is preferably 5˜50% (w/w), more preferably 7˜35% (w/w), still more preferably 9˜25% (w/w).

The preferable examples of butylene glycol for use in this invention are 1,3-butyleneglycol, 1,2-butyleneglycol, 2,3-butylene glycol, etc. The most preferable one is 1,3-butylene glycol.

The formulating amount of said butylene glycol is not particularly restricted, but may for example be 30˜60% (w/w), more preferably 40˜50% (w/w), most preferably 44˜46% (w/w).

The preferable examples of diethylene glycol mono(lower)alkyl ethers for use in this invention are diethylene glycol monoethyl ether, diethylene glycol monobutyl ether, etc. The most preferable one is diethylene glycol monoethyl ether.

The formulating amount of said diethylene glycol mono (lower)alkyl ethers is not particularly restricted, but may for example be 15˜60% (w/w), more preferably 20˜50% (w/w), most preferably 25˜45% (w/w).

The thickeners which is usable in this invention is not particularly restricted provided that it is pharmaceutically acceptable and capable of imparting viscosity to the pharmaceutical base, thus including the following organic and inorganic water-soluble macromolecular substances, among others.

(1) Organic Substances

Native polymers—gum Arabic, gum guar, carrageenan, gum tragacanth, pectin, starch, gum xanthan, gelatin, casein, dextrin, cellulose

Semisynthetic polymers—cellulose polymer (methylcellulose, ethylcellulose, hydroxyethylcellulose, hydroxypropylcellulose, carboxymethylcellulose sodium, carboxymethylcellulose calcium, etc.), carboxymethylstarch, sodium alginate, propylene glycol alginate

Synthetic polymers—carboxyvinyl polymer (Carbopol), polyvinyl alcohol, polyvinylpyrrolidone, polyethylene glycol, poly(vinyl methyl ether), sodium polyacrylate

(2) Inorganic Substances

Bentonite, synthetic magnesium silicate, magnesium aluminosilicate, silicon dioxide, etc.

The amount of the thickener in the pharmaceutical composition can be judiciously selected according to the objective viscosity of the pharmaceutical composition. For example, the thickener is used in a proportion of preferably 0.1˜10% (w/w), more preferably 0.5˜5% (w/w) . Among the specific examples given above, cellulose polymer such as hydroxypropylcellulose, carboxyvinyl polymer are more preferable, and the most preferable one is carboxyvinyl polymer. It is possible to change the touch of the pharmaceutical composition by changing them.

In addition to the above ingredients, the pharmaceutical composition of this invention may contain the conventional excipient (e.g. lactose, sucrose, starch, mannitol, etc.), stabilizer [antioxidant (e.g. ascorbyl palmitate, tocopherol, etc.)], coloring agent, sweetener, perfume, diluent and preservative, as well as other medicinally active substances.

The pharmaceutical composition of this invention can be used by applying it to the affected site, particularly the skin lesion, once to 4 times daily.

The proper amount of said tricyclic compounds in the pharmaceutical composition is dependent on its particular species used, the patient's age, the type of disease and its severity, and other factors. Typically, the recommended amount relative to the total composition is 0.00001˜20% (w/w), more preferably 0. 0001˜10% (w/w), most preferably 0.001˜3% (w/w). The composition may further contain one or more other drugs that are indicated in diseases of the skin.

Meanwhile, the pharmaceutical composition of this invention can be produced in the same manner as described in the following examples.

### EXAMPLES

The following examples are intended to illustrate this invention in further detail and should by no means be construed as defining the scope of the invention. In the following examples, FK506 substance is admixed as its monohydrate when preparing compositions containing it, though its amount is expressed as the weight of FK506 substance, not of its monohydrate.

### Example 1

### [Composition 1]

| | | |
|---|---|---|
| FK506 substance | 0.3% | (w/w) |
| Isopropyl myristate | 5.0% | (w/w) |
| Diethyl sebacate | 5.0% | (w/w) |
| Propylene carbonate | 5.0% | (w/w) |
| Diethylene glycol monoethyl ether | 37.5% | (w/w) |
| 1,3-Butylene glycol | 44.7% | (w/w) |
| Carboxyvinyl polymer (CP980NF) | 2.5% | (w/w) |
| Total | 100.0% | (w/w) |

FK506 Substance was dissolved with diethylene glycol monoethyl ether. And then 1,3-buthylene glycol and propylene carbonate were added. After visual check on the complete dissolution of FK506 substance, the dispersion of carboxyvinyl polymer in the binary system of isopropyl myristate and diethyl sebacate was added to obtain the desired viscosity and to provide a gel preparation for external application.

### Example 2

According to a similar manner to Example 1, the following pharmaceutical compositions 2, 3, 4 ,5 and 6 were prepared.

| | Composition No. | | | | |
|---|---|---|---|---|---|
| | 2 (% | 3 | 4 | 5 | 6 (% |
| | w/w) | (% w/w) | (% w/w) | (% w/w) | w/w) |
| FK506 substance | 0.3 | 0.3 | 0.3 | 0.3 | 0.3 |
| Isopropyl myristate | 5.0 | 5.0 | 5.0 | 5.0 | 5.0 |
| Diethyl sebacate | 10.0 | — | 5.0 | 10.0 | 5.0 |
| Diisopropyl adipate | — | 10 | 5.0 | 10.0 | 10.0 |
| Propylene carbonate | 5.0 | 5.0 | 5.0 | 7.5 | 7.5 |
| Diethylene glycol | 32.5 | 32.5 | 32.5 | 30.0 | 30.0 |
| monoethyl ether | | | | | |
| 1,3-Butylene glycol | 44.7 | 44.7 | 44.7 | 34.7 | 39.7 |
| Carboxyvinyl polymer | 2.5 | 2.5 | 2.5 | 2.5 | — |
| (CP980N F) | | | | | |
| Hydroxypropyl | — | — | — | — | 2.5 |
| cellulose | | | | | |
| Ascorbyl palmitate | — | — | — | — | 0.02 |

### Example 3

According to a similar manner to Example 1, the following pharmaceutical compositions 7 and 8 are prepared.

Composition No.

| | 7 | 8 |
|---|---|---|
| | (% w/w) | (% w/w) |
| Ascomycin | 0.3 | — |
| 33-epi-chloro-33-desoxy- | — | 0.3 |
| ascomycin | | |
| Isopropyl myristate | 5.0 | 5.0 |
| Diethyl sebacate | 5.0 | 5.0 |
| Diisopropyl adipate | 5.0 | 5.0 |
| Propylene carbonate | 5.0 | 5.0 |
| Diethylene glycol | 32.5 | 32.5 |
| monoethyl ether | | |
| 1,3-Butylene glycol | 44.7 | 44.7 |
| Carboxyvinyl polymer | 2.5 | 2.5 |
| (CP980NF) | | |

### Example 4

The percutaneous absorption experiments performed using the pharmaceutical composition of the invention are described below.

Using Composition Nos. 1 and 3 of Examples 1 and 2, an in vivo percutaneous absorption experiment was carried out.

As experimental animals, three 7-week-old male SD rats were used. With each animal immobilized in supine position in a stereotaxic device, the hair coat was removed with an electric clipper and the animal was returned to the cage and kept intact for 24 hours. After the animal was immobilized again in supine position in the stereotaxic device, a 2.5 cm×4 cm area was marked off on the depilated abdominal skin of the rat and 50 mg of the test drug was applied to said marked-off area. At predetermined times after medication, 0.3 ml of blood was withdrawn from the subclavian vein into an EDTA-containing syringe and, after through mixing of blood with EDTA, the blood sample was stored frozen until assayed. The whole blood concentration of FK506 Substance was determined by subjecting the blood sample to the enzyme immunoassay using a peroxidase (the assay system described in, for example, Japanese Kokai Tokkyo Koho H1-92659).

On the other hand, after blood sampling at the 24th hour, the surface of the medicated skin was washed with water and the skin tissue was excised from the above-mentioned marked-off area.

The percutaneous absorption parameters of the test drug were determined. The results are presented in Table 1. In Table 1, AUC [0˜24 hr] denotes the area under the 0˜24 hr blood concentration-time curve.

TABLE 1

| Sample | AUC [0-24 hr] |
|---|---|
| administered | (ng · hr/ml) |
| Composition 1 | >60 |
| Composition 3 | >60 |

### Example 5

### Stabilizing Test

The compositions 5 and 6 prepared in Example 2 were maintained 6 days at 70° C. and the remaining FK506 substance was calculated. The results are shown in Table 2.

TABLE 2

| Composition Nos. | Remaining FK506 |
|---|---|
| Composition 5 | >90% |
| Composition 6 | >90% |

It was also confirmed that carboxybinyl polymer could make FK506 stable, when FK506 was dissolved in lower alkanediols, such as ethylene glycol, propylene glycol, butylene glycol, etc. And, it was further confirmed that a combination of hydroxypropyl cellulose and ascorbyl palmitate could also make FK506 stable, when FK506 was dissolved in lower alkanediols, such as ethylene glycol, propylene glycol, butylene glycol, etc.

Therefore, the present application further provides (i) a new use of carboxybinyl polymer for stabilizing the tricyclic compound (1) which is dissolved in lower alkanediols and (ii) a new use of a combination of hydroxypropyl cellulose and ascorbyl palmitate for stabilizing the tricyclic compound (1) which is dissolved in lower alkanediols.

Effect of the Invention

In accordance with this invention there was provided a pharmaceutical composition containing the tricyclic compound (I) or its pharmaceutically acceptable salt, which is very satisfactory in stability, workability, user acceptance, irritation potential, less skin sensitization and/or dermal penetration efficiency. In particular, a gel preparation for external application could be provided which insures an improved penetration of the tricyclic compound (I) or its pharmaceutically acceptable salt, through the keratoid layer, which is a barrier to absorption, as well as a good cutaneous retention (particularly in the dermis) of the tricyclic compound. In addition, the pharmaceutical composition of this invention has an adequate emollient (humectant) action and is free from the risk for dermatrophy and the so-called rebound phenomenon.

The pharmaceutical composition of the present invention is useful for the treatment or prevention of inflammatory or hyperproliferative skin diseases or cutaneous manifestations of immunologically-mediated diseases (e.g. psoriasis, atopic dermatitis, contact dermatitis, eczematoid dermatitis, seborrheic dermatitis, lichen planus, pemphigus, bullous pemphigoid, epidermolysis bullosa, urticaria, angioedema, vasculitides, erythema, dermal eosinophilia, lupus erythematosus, acne, and alopecia areata) because of the pharmacologic activities possessed by the tricyclic compound. Particularly, the gel preparatrion for external use of the present invention is useful for the treatment or prophylaxis of psoriasis, such as psoriasis arthropathica, psoriasis circinata, psoriasis diffusa, psoriasis discoidea, generalized pustular psoriasis of Zumbusch, psoriasis geographica, psoriasis guttata, psoriasis gyrata, psoriasis inveterata, psoriasis nummularis, psoriasis orbicularis, psoriasis ostreacea, psoriasis punctata, pustular psoriasis, psoriasis spondylitica, psoriasis universalis, and so on.

Furthermore, the pharmaceutical composition of the present invention is also useful for the therapy or prophylaxis of the following diseases.

Autoimmune diseases of the eye (e.g. keratoconjunctivitis, vernal conjunctivitis, uveitis associated with Behcet's disease, keratitis, herpetic keratitis, conical keratitis, corneal epithelial dystrophy, keratoleukoma, ocular premphigus, Mooren's ulcer, scleritis, Graves' ophthalmopathy, Vogt-Koyanagi-Harada syndrome, keratoconjunctivitis sicca (dry eye), phlyctenule, iridocyclitis, sarcoidosis, endocrine ophthalmopathy, etc.);

skin diseases (e.g. dermatomyositis, leukoderma vulgaris, ichthyosis vulgaris, photosensitivity, and cutaneous T-cell lymphoma);

hypertrophic cicatrix or keloid due to trauma, burn, or surgery;

rejection reactions by transplantation of organs or tissues such as the heart, kidney, liver, bone marrow, skin, cornea, lung, pancreas, small intestine, limb, muscle, nerve, intervertebral disc, trachea, myoblast, cartilage, etc.;

graft-versus-host reactions following bone marrow transplantation;

autoimmune diseases such as rheumatoid arthritis, systemic lupus erythematosus, Hashimoto's thyroiditis, multiple sclerosis, myasthenia gravis, type I diabetes, etc.;

infections caused by pathogenic microorganisms (e.g. Aspergillus fumigatus, Fusarium oxysporum, Trichophyton asteroides, etc.);

reversible obstructive airways diseases [asthma (e.g. bronchial asthma, allergic asthma, intrinsic asthma, extrinsic asthma, and dust asthma), particularly chronic or inveterate asthma (e.g. late asthma and airway hyper-responsiveness) bronchitis, etc.]; mucosal or vascular inflammations (e.g. gastric ulcer, ischemic orthrombotic vascular injury, ischemic bowel diseases, enteritis, necrotizing enterocolitis, intestinal damages associated with thermal burns, leukotriene B4-mediated diseases);

intestinal inflammations/allergies (e.g. coeliac diseases, proctitis, eosinophilic gastroenteritis, mastocytosis, Crohn's disease and ulcerative colitis);

food-related allergic diseases with symptomatic manifestation remote from the gastrointestinal tract (e.g. migrain, rhinitis and eczema);

renal diseases (e.g. intestitial nephritis, Goodpasture's syndrome, hemolytic uremic syndrome, and diabetic nephropathy) nervous diseases (e.g. multiple myositis, Guillain-Barre syndrome, Meniere's disease, multiple neuritis, solitary neuritis, cerebral infarction, Alzheimer's disease, Parkinson's diseases, amyotrophiclateral sclerosis(ALS) and radiculopathy); cerebral ischemic disease (e.g., head injury, hemorrhage in brain (e.g., subarachnoid hemorrhage, intracerebral hemorrhage), cerebral thrombosis, cerebral embolism, cardiac arrest, stroke, transient ischemic attack (TIA), hypertensive encephalopathy, cerebral infarction) ;

endocrine diseases (e.g. hyperthyroidism, and Basedow's disease);

hematic diseases (e.g. pure red cell aplasia, aplastic anemia, hypoplastic anemia, idiopathic thrombocytopenic purpura, autoimmune hemolytic anemia, agranulocytosis, pernicious anemia, megaloblastic anemia, and anerythroplasia);

bone diseases (e.g. osteoporosis);

respiratory diseases (e.g. sarcoidosis, pulmonary fibrosis, and idiopathic interstitial pneumonia);

skin diseases (e.g. dermatomyositis, leukoderma vulgaris, ichthyosis vulgaris, photosensitivity, and cutaneous T-cell lymphoma);

circulatory diseases (e.g. arteriosclerosis, atherosclerosis, aortitis syndrome, polyarteritis nodosa, and myocardosis);

collagen diseases (e.g. scleroderma, Wegener's granuloma, and Sjogren's syndrome);

adiposis;

eosinophilic fasciitis;

periodontal diseases (e.g. damage to gingiva, periodontium, alveolar bone or substantia ossea dentis);

nephrotic syndrome (e.g. glomerulonephritis);

male pattern alopecia, alopecia senile;

muscular dystrophy;

pyoderma and Sezary syndrome;

chromosome abnormality-associated diseases (e.g. Down's syndrome);

Addison's disease;

active oxygen-mediated diseases [e.g. organ injury (e.g. ischemic circulation disorders of organs (e.g. heart, liver, kidney, digestive tract, etc.) associated with preservation, transplantation, or ischemic diseases (e.g. thrombosis, cardial infarction, etc.)):

intestinal diseases (e.g. endotoxin shock, pseudomembranous colitis, and drug- or radiation-induced colitis):

renal diseases (e.g. ischemic acute renal insufficiency, chronic renal failure):

pulmonary diseases (e.g. toxicosis caused by pulmonary oxygen or drugs (e.g. paracort, bleomycin, etc.), lung cancer, and pulmonary emphysema):

ocular diseases (e.g. cataracta, iron-storage disease (siderosis bulbi), retinitis, pigmentosa, senile plaques, vitreous scarring, corneal alkali burn):

dermatitis (e.g. erythema multiforme, linear immunoglobulin A bullous dermatitis, cement dermatitis):

and other diseases (e.g. gingivitis, periodontitis, sepsis, pancreatitis, and diseases caused by environmental pollution (e.g. air pollution), aging, carcinogen, metastasis of carcinoma, and hypobaropathy)];

diseases caused by histamine release or leukotriene C4 release;

restenosis of coronary artery following angioplasty and prevention of postsurgical adhesions;

autoimmune diseases and inflammatory conditions (e.g., primary mucosal edema, autoimmune atrophic gastritis, premature menopause, male sterility, juvenile diabetes mellitus, pemphigus vulgaris, pemphigoid, sympathetic ophthalmitis, lens-induced uveitis, idiopathic leukopenia, active chronic hepatitis, idiopathic cirrhosis, discoid lupus erythematosus, autoimmune orchitis, arthritis (e.g. arthritis deformans), or polychondritis);

Human Immunodeficiency Virus (HIV) infections AIDS;

allergic conjunctivitis;

hypertrophic cicatrix and keloid due to trauma, burn, or surgery.

In addition, the said tricyclic compound (I) has liver regenerating activity and/or activities of stimulating hypertrophy and hyperplasia of hepatocytes. Therefore, the pharmaceutical composition of the present invention is useful for increasing the effect of the therapy and/or prophylaxis of liver diseases [e.g. immunogenic diseases (e.g. chronic autoimmune liver diseases such as autoimmune hepatic diseases, primary biliary cirrhosis or sclerosing cholangitis), partial liver resection, acute liver necrosis (e.g. necrosis caused by toxins, viral hepatitis, shock, or anoxia), hepatitis B, non-A non-B hepatitis, hepatocirrhosis, and hepatic failure (e.g. fulminant hepatitis, late-onset hepatitis and “acute-on-chronic” liver failure (acute liver failure on chronic liver diseases))]. Particularly, it is preferable to treat or prevent hepatitis, such as chronic hepatitis C, by applying the tricyclic compounds (I) together with various interferons.

And further, the present composition is also useful for increasing the effect of the prevention and/or treatment of various diseases because of the useful pharmacological activity of the said tricyclic compounds (I), such as augmenting activity of chemotherapeutic effect, activity of cytomegalovirus infection, anti-inflammatory activity, inhibiting activity against peptidyl-prolyl isomerase or rotamase, antimalarial activity, antitumor activity, and so on.

The disclosure of the patents, patent applications and references cited herein in the present application is encompassed within the description of the present specification.

1. A pharmaceutical composition, comprising a tricyclic compound of the formula (i): 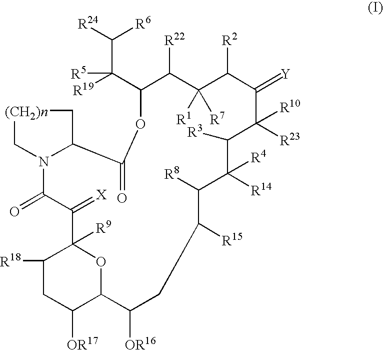
wherein each of adjacent pairs of R¹ and R², R³ and R⁴═, and R⁵ R⁶ independently
(a) is two adjacent hydrogen atoms, or R¹ is a hydrogen atom and R² is an alkyl group or
(b) form another bond between the carbon atoms to which they are attached;
R⁷ is a hydrogen atom, a hydroxy group, a protected hydroxy group, or an alkoxy group, or an oxo group together with R¹;
R⁸ and R⁹ are independently a hydrogen atom or a hydroxy group;
R¹⁰ is a hydrogen atom, an alkyl group, an alkyl group substituted by one or more hydroxy groups, an alkenyl group, an alkenyl group substituted by one or more hydroxy groups, or an alkyl group substituted by an oxo group;
X is an oxo group, (a hydrogen atom and a hydroxy group), (a hydrogen atom and a hydrogen atom), or a group represented by the formula —CH₂O—;
Y is an oxo group, (a hydrogen atom and a hydroxy group), (a hydrogen atom and a hydrogen atom), or a group represented by the formula N—NR¹¹R¹² or N—OR¹³;
R¹¹ and R¹² are independently a hydrogen atom, an alkyl group, an aryl group or a tosyl group;
R¹³, R¹⁴, R¹⁵, R¹⁶, R¹⁷, R¹⁸, R¹⁹, R²² and R²³ are independently a hydrogen atom or an alkyl group;
R²⁴ is an optionally substituted ring system containing one or more heteroatoms;
n is an integer of 1 or 2; and
in addition to the above definitions, Y, R¹⁰ and R²³, together with the carbon atoms to which they are attached, are a saturated or unsaturated 5- or 6-membered nitrogen, sulfur and/or oxygen containing heterocyclic ring optionally substituted by one or more groups selected from the group consisting of an alkyl, a hydroxy, an alkoxy, a benzyl, a group of the formula —CH₂Se(C₆H₅), and an alkyl substituted by one or more hydroxy groups, or its pharmaceutically acceptable salt;
at least one monohydric alcohol fatty acid ester;
at least one dibasic acid diester;
at least one lower alkylene carbonate;
butylene glycol;
at least one diethylene glycol mono(lower)alkyl ether; and
at least one thickener.

2. The pharmaceutical composition according to claim 1, comprising the tricyclic compound (I) wherein each of adjacent pairs of R³ and R⁴ or R⁵ and R⁶ independently form another bond between the carbon atoms to which they are attached;
each of R⁸ and R²³ is independently a hydrogen atom;
R⁹ is a hydroxy group;
R¹⁰ is a methyl group, an ethyl group, a propyl group or an allyl group;
X is (a hydrogen atom and a hydrogen atom) or an oxo group;
Y is an oxo group;
each of R¹⁴, R¹⁵, R¹⁶, R¹⁷, R¹⁸, R¹⁹, and R²² is a methyl group;
R²⁴ is a 3-R²⁰-4-R²¹-cyclohexyl group,
in which R²⁰ is hydroxy, an alkoxy group, an oxo group, or a —OCH₂OCH₂CH₂OCH₃ group, and
R²¹ is hydroxy, —OCN, an alkoxy group, a heteroaryloxy which is substituted by suitable substituents, 1- or 2-tetrazolyl, a —OCH₂OCH₂CH₂OCH₃ group, a protected hydroxy group, chloro, bromo, iodo, aminooxalyloxy, an azido group, p-tolyloxythiocarbonyloxy, or R²⁵R²⁶CHCOO—,
in which R²⁵ is optionally protected hydroxy or protected amino, and
R²⁶ is hydrogen or methyl, or
R²⁰ and R²¹ together form an oxygen atom in an epoxide ring; and
n is an integer of 1 or 2.

3. The pharmaceutical composition according to claim 1, wherein said tricyclic compound (I) is 17-allyl-1,14-dihydroxy-12-[2-(4-hydroxy-3-methoxycyclohexyl)-1-methylvinyl]-23,25-dimethoxy-13,19,21,27-tetramethyl-11,28-dioxa-4-azatricyclo[22.3.1.0^{4,9}]octacos-18-ene-2,3,10,16-tetraone or its hydrate.

4. The pharmaceutical composition according to claim 1 wherein the monohydric alcohol fatty acid ester is isopropyl myristate; the dibasic acid diester is diisopropyl adipate and/or diethyl sebacate; and the lower alkylene carbonate is propylene carbonate.

5. The pharmaceutical composition according to claim 1, wherein the diethylene glycol mono(lower)alkyl ether is diethylene glycol monoethyl ether.

6. The pharmaceutical composition according to claim 1, wherein each amount of monohydric alcohol fatty acid ester, dibasic acid diester and lower alkylene carbonate is 1-30% (w/w), respectively.

7. The pharmaceutical composition according to claim 1, wherein the amount of diethylene glycol mono(lower)alkyl ether is 15-60% (w/w).

8. The pharmaceutical composition according to claim 1, wherein the amount of butylene glycol is 30-60% (w/w).

9. The pharmaceutical composition according to claim 1, wherein the thickener is a carboxyvinyl polymer.

10. A method for stabilizing a tricyclic compound comprising combining a carboxyl polymer with the tricyclic compound of the formula (I) according to claim 1 dissolved in at least one alkanediol.

11. A method for stabilizing a tricyclic compound comprising combining a combination of hydroxy propyl cellulose and ascorbyl palmitate with the tricyclic compound of formula I according to claim 1 dissolved in at least one alkanediol.
